(19) United States
(12) Patent Application Publication
(10) Pub. No.: US 2013/0267955 A1
(43) Pub. Date: Oct. 10, 2013

(54) SYSTEMS FOR CONTROLLING BONE GROWTH

(71) Applicant: UNIVERSITY OF UTAH RESEARCH FOUNDATION, Salt Lake City, UT (US)

(72) Inventors: Erik N. KUBIAK, Salt Lake City, UT (US); Charles L. SALTZMAN, Salt Lake City, UT (US)

(73) Assignee: UNIVERSITY OF UTAH RESEARCH FOUNDATION, Salt Lake City, UT (US)

(21) Appl. No.: 13/830,456

(22) Filed: Mar. 14, 2013

Related U.S. Application Data
(63) Continuation-in-part of application No. 13/178,460, filed on Jul. 7, 2011.
Application No. 13/178,460 is a continuation-in-part of application No. 12/577,688, filed on Oct. 12, 2009.
Application No. 12/577,688 is a continuation-in-part of application No. 12/577,683, filed on Oct. 12, 2009.

(51) Int. Cl. A61B 17/80

(52) U.S. Cl. CPC A61B 17/80
USPC 606/70; 606/281

(57) ABSTRACT

Systems and methods for stabilizing portions of bone are provided. In some aspects, a bone fixation system can include a plate configured to be fastened to a growing bone to control growth of the bone in at least one direction. The system can include a keel structure having a longitudinal axis. The keel can extend from the plate into the bone to permit bone growth along the keel longitudinal axis on one side of the bone while the plate generally inhibits growth on the other side of the bone.

### RELATED APPLICATIONS

This application is a continuation-in-part of U.S. patent application Ser. No. 13/178,460, entitled “Bone Plate and Keel Systems,” filed on Jul. 7, 2011, which is a continuation-in-part of U.S. patent application Ser. No. 12/577,688, entitled “Bone Fixation Systems,” filed on Oct. 12, 2009, and a continuation-in-part of U.S. patent application Ser. No. 12/577,683, entitled “Bone Fixation and Compression Systems,” filed on Oct. 12, 2009, each of which are hereby incorporated by reference in their entirety for all purposes.

### FIELD

Some embodiments of the present inventions generally relate to bone stabilization and, in particular, relate to systems and methods for stabilizing portions of bone.

### BACKGROUND

Bone plates are surgical tools used to assist in the healing of bones that are fractured or have undergone surgery. For example, fractures may be set and held in place using bone plates. Bone plates can be applied to fractures occurring in many bones throughout the skeleton, such as in the spine.

### SUMMARY

The present disclosure provides bone stabilizing methods and apparatuses for stabilizing and aligning portions of bone of a person whose bones are growing. The system may comprise a plate that is configured to be fastened to a bone, such as at the diaphysis, metaphysis, and/or across the physis to attach to the epiphysis.

For example, in some embodiments, the plate can be affixed to two bone portions (e.g., first and second bone portions) separated by a growth plate, epiphyseal plate, or physis. The system may comprise a keel structure projecting from the plate. For example, the keel structure can extend from a bone engaging surface of the plate. The keel structure may comprise first and second keels that may extend into the two bone portions separated by the physis in order to align the two bone portions when the plate is fastened to the bone portions.

The methods and apparatuses disclosed herein can aid in realigning limbs of children, e.g., by affixing a plate to the diaphysis, metaphysis, and/or across the physis to the epiphysis. Although generally, instead of growing from the center outward, long bones grow at each of their ends by virtue of tissue growth and ossification around the growth plate, methods provided herein enable a plate to be affixed only in the diaphysis region of the bone, only in the metaphysis region of the bone, across both the diaphysis and metaphysis regions of the bone, or across the metaphysis, the physis, and ephiphysis regions of the bone. Any of the long bones of the body can be targeted using the systems and methods disclosed herein, such as the femur, humerus, radius, ulna, tibia, and fibula. These methods and apparatuses can be used to align portions of long bones, correcting issues including, for example, cubitus varus, knee valgus, knee varus, oblique plane, flexion deformity, recurvatum, and ankle valgus.

According to an aspect of at least one of the embodiments disclosed herein is the realization that previous options to realign of bones and joints do not allow for predictable and reliable control of bone growth in all directions. Some options, such as staples, tend to prevent growth on a side of the bone being restrained and fail to inhibit rotation or angulation of the bone portions relative to each other.

Therefore, the present disclosure provides several methods and apparatuses that overcome these challenges and allow a clinician to correct bone and joint alignment while controlling the rotation and angulation of respective bone portions. The methods and apparatuses disclosed herein can also allow the clinician to control limit anteroposterior growth and/or rotation.

Further, the methods and apparatuses can provide a plate having one or more holes. The plate can be configured such that the fasteners can move, rock, pivot, and/or slide relative to the plate as the bone grows. For example, after the fasteners are inserted into the bone and the bone grows, the orientation of the fasteners relative to the plate can change. Although in non-slotted embodiments, the head of the fastener may remain generally stably positioned relative to the plate, the body of the fastener can move relative to the plate. Relative to each other, the fasteners can separate or diverge like a hinge, allowing the growing bone to grow more rapidly on one side of the bone. This hinge action allows the plate to generally permit growth on the unrestrained side of the bone (opposite the side on which the plate is applied) while compressing or restraining growth on the side of the bone to which the plate is applied.

For example, a curvature in a bone can be reduced and/or eliminated to correct bone deformity. In order to do so, the plate can be placed on the side of the bone that bows outward or appears convex, such that the opposite side (which bows inwardly or appears concave) can then grow in the direction of the keel longitudinal axis to promote growth of the bone toward a linear or straight axial configuration. Thereafter, normal bone growth can resume on both sides of the bone.

Furthermore, in some embodiments, the plate can have one or more longitudinal slots for receiving a fastener that allow the plate to move relative to first and second bone portions as the physis produces additional bone growth, thus allowing a fastener attached to a bone portion to slide in the slots as the bone grows.

In some embodiments, a method is provided for controlling growth of a growing bone. The bone can have a first side and a second side opposite the first side. The method can comprise applying a bone fixation plate to the first side of the bone. The plate can comprise at least one keel having a longitudinal axis. The plate can be applied such that when affixed to the bone, linear bone growth occurs on the second side of the bone in the direction of the keel longitudinal axis while bone growth is inhibited on the first side.

At the time the plate is applied to the bone, the bone can comprise a curvature and the plate can be configured to permit growth along the second side of the bone to change the bone curvature.

The plate can be applied to a metaphysis of the bone. The plate can also be applied to a diaphysis of the bone.

The keel can comprise first and second keel portions, and the bone can comprise a physis. The method can further comprise applying the plate such that the first and second keel portions are positioned on opposing sides of the physis.

In some embodiments, a method is provided for stabilizing first and second bone portions of a person whose bones are growing. The first and second bone portions can be separated by a physis. The method can comprise: positioning a bone fixation plate to extend from the first bone portion to the second bone portion across the physis; affixing a first portion of the plate to the first bone portion, the plate comprising a first keel portion extending into the first bone portion; and affixing a second portion of the plate to the second bone portion, the plate comprising a second keel portion extending into the second bone portion, the first and second keels defining a keel longitudinal axis, wherein when affixed to the bone, the plate is configured to permit growth in the direction of the keel longitudinal axis.

The method can be performed such that affixing the plate first portion to the first bone portion comprises inserting a screw through a first hole formed in the first portion. Further, affixing the plate second portion to the second bone portion can comprise inserting a screw through a second hole formed in the second bone portion.

The method can be performed such that positioning the bone fixation plate comprises orienting the first and second keel portions to extend generally along the longitudinal axis of the bone such that the plate limits growth in the direction of the bone longitudinal axis.

The method can also be performed such that affixing the plate first portion to the first bone portion comprises extending the first keel portion into only the cortex of the first bone portion. Further, affixing the plate second portion to the second bone portion can comprise extending the second keel portion into only the cortex of the second bone portion.

Some embodiments can also provide a method for preparing first and second bone portions of a person whose bones are growing, the first and second bone portions having a physis therebetween. The method can comprise: affixing a plate to the first bone portion, the plate comprising a first hole at a first portion of the plate and a second hole at a second portion of the plate, the plate further comprising first and second keel slots; inserting a first fastener through the first opening into the first bone portion; inserting a second fastener through the second opening into the second bone portion; cutting bone through the first and second keel slots to form first and second grooves in the first and second bone portions.

The method can also comprise inserting the first and second keel portions substantially within the first and second grooves and spans the physis. The method can further comprise progressively advancing the first fastener such that the first and second bone portions become aligned with each other and progressively compress against each other.

A bone fixation system is also provided in some embodiments. The system aid in stabilizing first and second portions of a bone separated by a physis. The system can comprise a bone fixation plate having first and second portions configured to extend across the physis and be fastened to the first and second bone portions. Further, the system can comprise a split keel structure having a first keel portion extending from the plate first portion and a second keel portion extending from the plate second portion. The second keel portion can be separate from the first keel portion. The first and second keel portions can be configured to extend into the first and second bone portions.

In some embodiments, the first and second keel portions can be interconnected by an intermediate ridge. The system can be configured such that the first and second keel portions converge toward each other and taper toward the plate. Further, the first and second keel portions can be oriented parallel relative to each other.

The system can comprise a first protrusion extending transversely relative to and adjacent to the first keel portion. The system can also comprise a second protrusion extending transversely relative to and adjacent to the second keel portion.

The plate can comprise a first keel slot, and the system can further comprise a locking assembly. The first keel slot can be configured to engage with the first keel portion to prevent the first keel portion from dislodging from the plate when the first keel portion extends into the first bone portion through the first keel slot. The plate can further comprise a second keel slot, the second keel slot configured to engage the second keel portion to prevent the second keel portion from dislodging from the plate when the second keel portion extends into the second bone portion through the second keel slot.

In some embodiments, the first keel portion can extend from the plate and be configured to stabilize the plate relative to the first bone portion. The second keel portion can extend from the plate and be configured to stabilize the plate relative to the second bone portion.

The first keel portion can have a longitudinal axis extending generally parallel relative to a longitudinal axis of the plate. Further, the second keel portion can have a longitudinal axis extending generally parallel relative to a longitudinal axis of the plate. In addition, the system can comprise a first protrusion extending transversely relative to the first keel portion and a second protrusion extending transversely relative to the second keel portion.

A ratio of an average thickness of the plate to an average width of the plate may be less than or equal to about 0.11. In some aspects, the ratio may be less than or equal to about 0.10. In some aspects, the ratio may be less than or equal to about 0.09. In some aspects, the ratio may be less than or equal to about 0.08.

In certain aspects, the plate comprises a first hole in a first portion of the plate. The first hole and the plate may be configured such that a first fastener extends through the first hole and fastens the plate to a first bone portion. The plate may comprise a second hole in a second portion of the plate. The second hole may be configured to receive a second fastener therethrough for fastening the plate to the bone portions.

In some aspects, the plate and the keel structure may be integrally formed. The plate may be substantially orthogonally coupled to the keel structure (e.g., within a range of 80-100 degrees). At least one of the plate or the keel structure may comprise at least one of steel or titanium. In some aspects, the keel structure may comprise porous metal.

According to certain aspects, the system may further comprise a first protrusion projecting from a first half of the bone engaging surface and configured such that when the plate is fastened to the bone portions, the first protrusion extends into at least one of the bone portions. The system may further comprise a second protrusion projecting from a second half of the bone engaging surface and configured such that when the plate is fastened to the bone portions, the second protrusion extends into at least another of the bone portions.

The system may further comprise a jig having a first slot permitting cutting of bone through the first slot. The jig can also comprise a second slot spaced apart from the first slot such that the first and second slots are positioned on opposing sides of the physis when the jig is fastened to the bone portions.

According to various aspects of the present disclosure, the bone portions can be stabilized by cutting a first bone portion to form a first groove in the first bone portion and cutting a second bone portion to form a second groove in the second bone portion. A physis of the bone can be positioned between the first and second bone portions. The method may also comprise fastening a plate to the bone portions such that a keel structure of the plate fits substantially within the first and second grooves. The method can be performed without damaging the physis of the bone.

In some aspects, the plate may comprise a first hole at a first portion of the plate. The fastening the plate may comprise inserting a first fastener through the first hole into the first bone portion to fasten the plate first portion to the first bone portion. In some aspects, the method may further comprise drilling bone through a first location corresponding to the first hole to form a first channel. The first fastener may fit substantially within the first channel.

In some aspects, the plate may further comprise a second hole at a second portion of the plate. The fastening the plate may further comprise inserting a second fastener through the second hole into at least one of the bone portions. In some aspects, the method may further comprise drilling bone through a second location corresponding to the second hole to form a second channel. The second fastener may fit substantially within the second channel.

In some aspects, the method may further comprise fastening a jig to the bone portions. The jig may comprise a first slot permitting cutting of bone through the first slot and a second slot permitting cutting of bone through the second slot. The first and second slots may be spaced apart to span the physis when the jig is fastened to the bone portions. The cutting may comprise cutting bone through the first slot to form a first groove and cutting bone through the second slot to form a second groove.

In some aspects, the jig may comprise a second slot permitting cutting of bone through the second slot. The method may further comprise cutting bone through the second slot to form a second groove in at least one of the bone portions. The fastening the plate may comprise fastening the plate to the bone portions such that a protrusion from the plate fits substantially within the second groove.

The system may also comprise a fin projecting from a keel structure. The fin may be configured such that as the plate is fastened to the first and second bone portions, the fin (a) is inserted into a bone portion and thereafter (b) is progressively urged into the bone portion.

In some aspects, the fin may further be configured to align and progressively compress the first and second bone portions against each other.

In some aspects, a long axis of the fin and a long axis of the plate may be substantially non-parallel. The fin may further be configured such that (a) as the plate is fastened to a bone portion and (b) as the fin is progressively advanced into the bone portion, the fin moves in a direction away from a contact surface of the bone portion and away from the plate.

In some aspects, the system further comprises a liner configured to substantially prevent contact between the plate and the keel structure. In some aspects, the liner comprises polyether ether ketone (PEEK). In some aspects, the locking assembly comprises a liner positioned in the keel structure slot. The liner is configured to substantially prevent contact between the plate and the keel structure. In some aspects, the liner is configured to conform to a surface of the plate and to a surface of the keel structure.

According to various aspects of the present disclosure, bone portions can also be stabilized by fastening a plate to first and second bone portions, the plate having a pair of keel slots and locking assemblies; extending a keel structure into each bone portion through each keel slot; and substantially preventing, with a respective locking assembly, the keel structure from dislodging from the plate when the keel structure extends into the bone portion through the keel slot.

In some aspects, the locking assembly can comprise (i) a first lip region extending along a first wall of the keel slot and (ii) a second lip region extending along a second wall of the keel slot. The second wall is opposite the first wall. The method can further comprise flexing the locking assembly between a first state and a second state, wherein the first and second lip regions are farther apart from one another in the first state than when the first and second lip regions are in the second state.

According to various aspects of the present disclosure, bone portions can also be stabilized by fastening a plate to at least one bone portion in which a keel structure of the plate extends from the plate in a direction away from the bone portion when the plate is fastened to the bone portion.

Additional features and advantages of the invention will be set forth in the description below, and in part will be apparent from the description, or may be learned by practice of the invention. The advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide further understanding of the invention and are incorporated in and constitute a part of this specification, illustrate aspects of the invention and together with the description serve to explain the principles of the invention.

FIGS. 1A, 1B, and 1C illustrate a bone plate system, according to some embodiments.

FIGS. 2A and 2B illustrate another bone plate system, according to some embodiments.

FIGS. 3A and 3B illustrate an example of a bone plate system with one or more protrusions, according to some embodiments.

FIGS. 4A and 4B illustrate a jig system used for fastening a plate to bone portions, according to some embodiments.

FIGS. 5A and 5B illustrate a bone plate system with a plate fastened to bone portions, according to some embodiments.

FIG. 5C illustrate a bone plate, according to some embodiments.

FIG. 5D illustrates a bone and a plurality of bone plate positions, according to some embodiments.

FIGS. 6A-6C illustrate various configurations of a plate and a keel structure, according to some embodiments.

FIG. 7A illustrates a perspective view of a bone plate system, according to some embodiments.

FIG. 7B illustrates a top view of the bone plate system of FIG. 7A positioned against bone portions, according to some embodiments.

FIG. 7C illustrates a side view of a bone plate system, according to some embodiments.

FIGS. 7D-7E illustrate examples of a keel structure, according to some embodiments.

FIG. 8A illustrates an example of a locking assembly, according to some embodiments.

FIG. 8B illustrates another example of a locking assembly, according to some embodiments.

FIG. 9 illustrates yet another example of a locking assembly, according to some embodiments.

FIGS. 10A-10C illustrate another example of a locking assembly, according to some embodiments.

FIGS. 11A-11D illustrate various views of a liner that can be used to prevent contact between a keel and a plate, according to some embodiments.

FIGS. 12A-12C illustrate various views of another liner that can be used to prevent contact between a keel and a plate, according to some embodiments.

### DETAILED DESCRIPTION

In the following detailed description, numerous specific details are set forth to provide a full understanding of the present invention. It will be apparent, however, to one ordinarily skilled in the art that the present invention may be practiced without some of these specific details. In other instances, well-known structures and techniques have not been shown in detail so as not to obscure the present invention.

According to various aspects of the present disclosure, systems and methods are provided for stabilizing portions of growing bone, for example, by fixing a desired orientation between first and second bone portions that are separated by a growth plate, epiphyseal plate, or physis.

The present disclosure is related to U.S. patent application Ser. No. 13/178,460, entitled “Bone Plate and Keel Systems,” filed on Jul. 7, 2011, U.S. patent application Ser. No. 12/577,688, entitled “Bone Fixation Systems,” filed on Oct. 12, 2009, and U.S. patent application Ser. No. 12/577,683, entitled “Bone Fixation and Compression Systems,” filed on Oct. 12, 2009, each of which is hereby incorporated by reference herein in its entirety for all purposes.

Bone plates may be used in orthopedic surgery as a means to affix bones to each other to promote healing. One disadvantage of these plates, for example in relation to the use of these plates in foot surgery, is in their thickness profile. The extreme thickness of these plates may result in soft tissue irritation in the area surrounding the plate. Unfortunately, the thickness of these plates may relate directly to their strength. Therefore, thickness of these plates may not be modified without affecting the strength of the plates. Reducing the thickness of the plate, in an attempt to reduce soft tissue irritation may lead to plate failure.

Some embodiments disclosed herein can provide a plate that may incorporate a “keel” structure along its bottom or top surface. The keel structure can comprise at least one keel. For example, the keel structure can comprise two keels that are separate or formed from a continuous piece of material. The keel structure may provide additional strength to the plate structure, thereby allowing the thickness to be reduced. Further, the keel structure can serve to control or limit growth in a desired direction, such as in the longitudinal direction of the bone. In some aspects, fasteners may be used to attach the plate to bone portions for increased stabilization. The plate may control rotation, anteroposterior movement, facilitate linear compression, and/or minimize soft tissue irritation of the first and second bone portions.

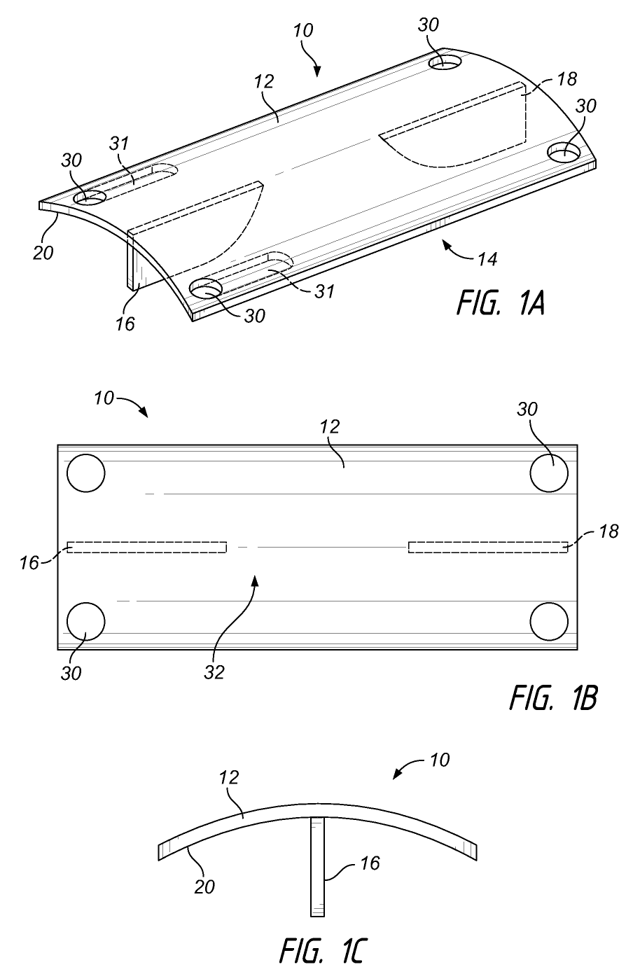

FIGS. 1A, 1B, and 1C illustrate an example of a bone plate system 10, in accordance with various aspects of the present disclosure. FIG. 1A shows a side view of bone plate system 10, FIG. 1B shows a top view of bone plate system 10, and FIG. 1C shows a front view of bone plate system 10.

The bone plate system 10 may comprise a plate 12 and a keel structure 14. In the illustrated embodiment, the keel structure 14 comprises a first or upper keel 16 and a second or lower keel 18. The plate 12 can have a bone engaging surface (e.g., the bottom side of the plate 12 as shown in FIGS. 1A and 1C). The first and second keels 16, 18 can extend from the bone engaging surface 20.

As discussed further herein, the plate 12 may be fastened to first and second bone portions having a growth plate, epiphyseal plate, or physis positioned therebetween, for example, for controlling direction and orientation of growth of bone between the first and second bone portions. The keel structure 14 may project from the bone engaging surface and extend into the first and second bone portions and span the physis when the plate 12 is fastened to the first and second bone portions.

The plate 12 may also comprise one or more holes 30 such that the one or more holes 30 may receive one or more fasteners therethrough for fastening the plate 12 to the bone portions. The one or more holes 30 may be counter-sunk (e.g., such that heads of fasteners may lie below a surface of the plate 12). The fasteners (not shown) can comprise one or more k wires, screws, nails, or other suitable fasteners known to those of skill in the art.

In accordance with some embodiments, the plate 12 can be configured such that the fasteners can move, rock, pivot, and/or slide relative to the plate 12 as the bone grows. For example, after the fasteners are inserted into the bone and the bone grows, the orientation of the fasteners relative to the plate can change. Although in non-slotted embodiments, the head of the fastener may remain generally stably positioned relative to the plate, the body of the fastener can move relative to the plate. Relative to each other, the fasteners can separate or diverge like a hinge, allowing the growing bone to grow more rapidly on one side of the bone. This hinge action allows the plate to generally permit growth on the unrestrained side of the bone (opposite the side on which the plate is applied) while compressing or restraining growth on the side of the bone to which the plate is applied.

For example, a curvature in a bone can be reduced and/or eliminated to correct bone deformity. In order to do so, the plate can be placed on the side of the bone that bows outward or appears convex, such that the opposite side (which bows inwardly or appears concave) can then grow in the direction of the keel longitudinal axis to promote growth of the bone toward a linear or straight axial configuration. Thereafter, normal bone growth can resume on both sides of the bone.

Further, although the one or more holes 30 can be round, the one or more holes 30 can be configured as slots that allow the fasteners to move therewithin in order to allow the fasteners to move relative to the plate 12 as the bone grows. A slot can be used instead of a round hole in any of the embodiments disclosed herein. An example pair of slots 31 are illustrated in FIG. 1A and can be applied in any of the embodiments disclosed herein.

In some aspects, the plate 12 and the keel structure 14 may be integrally formed. In some aspects, the plate 12 and the keel structure 14 may be separately formed.

As shown in FIG. 1A, the keel structure 14 can comprise at least one keel. For example, the keel structure can comprise separate or individual first and second or first and second keels 16, 18. The first and second keels 16, 18 can be spaced apart from each other along a longitudinal axis of the plate 12.

As illustrated in FIG. 1B, a gap 32 can be formed between the first and second keels 16, 18. The gap 32 can be configured to traverse or extend across the physis when the plate 12 is attached to the first and second bone portions. Accordingly, in such embodiments, the plate 12 can extend adjacent to the physis without contacting or causing any damage or otherwise engaging the physis.

In some aspects, the plate 12 may be substantially orthogonally coupled to the keel structure 14 (e.g., as seen in a side view of bone plate system 10 in FIG. 1C). The plate 12 may be curved or flat. At least one of the plate 12 or the keel structure 14 may comprise at least one of steel or titanium. In some aspects, the keel structure 14 may comprise an ingrowth material to achieve further stability and encourage bone growth. The keel structure 14 may comprise porous metal.

For example, the keel structure may comprise at least one of trabecular metal (e.g., tantalum spray on a styrofoam substrate) or biofoam (e.g., titanium on a similar substrate). The keel structure 14 may comprise any material known to those of skill in the art to form an osteointegrative surface thereon (e.g., surface for bone integration).

The keel structure 14 may increase the torsional stiffness as compared with traditional plates. The inclusion of the keel structure 14 in bone plate system 10 may allow the plate 12 to have a thinner profile. In some aspects, the average length of the plate 12 may be greater than the average width of the plate 12, which may be greater than the average thickness of the plate 12. Thus, the inclusion of the keel structure 14 in bone plate system 10 may reduce a ratio between the average thickness of the plate 12 to an average width of the plate 12 compared to traditional plates without the keel structure 14.

In some aspects, a ratio of an average thickness of the plate 12 to an average width of the plate 12 is less than or equal to about 0.11. In some aspects, a ratio of an average thickness of the plate 12 to an average width of the plate 12 is less than or equal to about 0.10. In some aspects, a ratio of an average thickness of the plate 12 to an average width of the plate 12 is less than or equal to about 0.09. In some aspects, a ratio of an average thickness of the plate 12 to an average width of the plate 12 is less than or equal to about 0.08. In some aspects, a ratio of an average thickness of the plate 12 to an average width of the plate 12 is less than or equal to about 0.07. In some aspects, a ratio of an average thickness of the plate 12 to an average width of the plate 12 is less than or equal to about 0.06. In some aspects, a ratio of an average thickness of the plate 12 to an average width of the plate 12 is less than or equal to about 0.05. In some aspects, a ratio of an average thickness of the plate 12 to an average width of the plate 12 is less than or equal to about 0.04. In some aspects, a ratio of an average thickness of the plate 12 to an average width of the plate 12 is less than or equal to about 0.03.

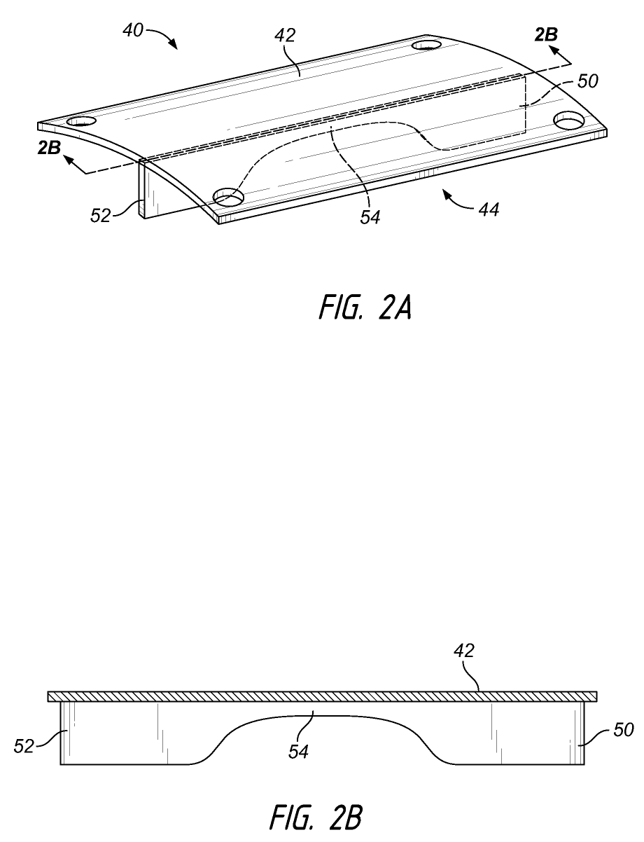

FIGS. 2A-B illustrate another embodiment of the bone plate system in which the keel structure comprises first and second keels that are interconnected or continuous. FIG. 2A is a perspective view of a bone plate system 40 having a bone plate 42 and a keel structure 44. FIG. 2B is a cross-sectional side view of FIG. 2A and illustrates that the keel structure 44 can have first and second keels 50, 52 that are interconnected by a ridge portion 54. Thus, in some embodiments, the keel structure 44 can extend continuously between first and second keels 50, 52 along a longitudinal length of the plate 40. However, similar to the embodiment illustrated in FIGS. 1A-C, the keel structure 44 of the bone plate system 40 can also define a reduced profile along a middle portion thereof (shown as ridge portion 54). This reduced profile portion can be used to straddle the physis in order to avoid substantial or significant interference with the physis while providing continuity of vertical alignment between the first and second bone portions.

Other features and aspects of the bone plate system 40 can be configured similar to the bone plate system 10 discussed above and will not be repeated here for brevity.

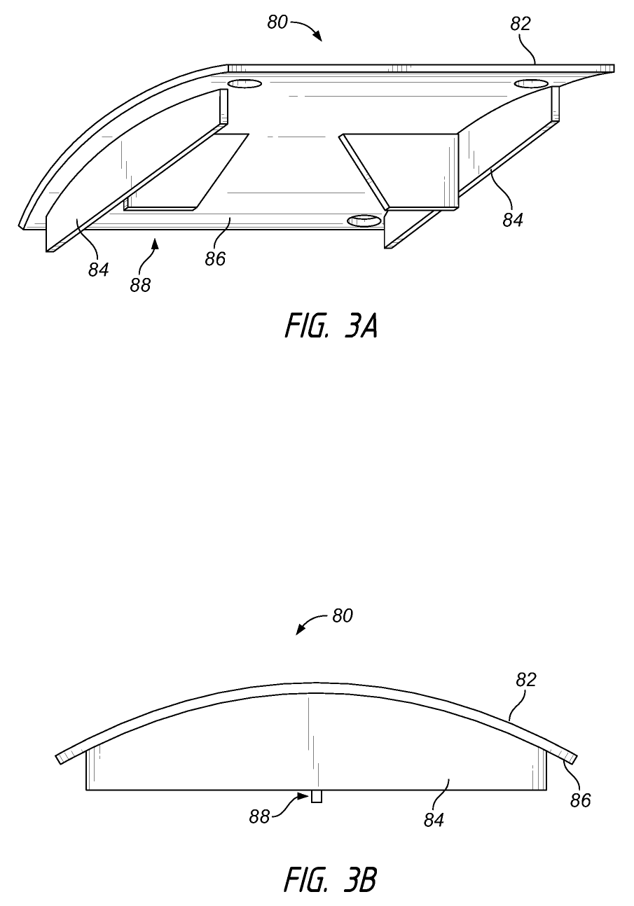

FIGS. 3A-B illustrate another embodiment of the bone plate system. In this embodiment, a bone plate system 80 can be provided that comprises a plate 82, one or more protrusions 84 protruding from a bone engaging surface 86 of the plate 82, and a keel structure 88 having two separate or interconnected keels. The one or more protrusions 84 may extend into at least one of the bone portions when the plate 82 is fastened to the bone portions. The system can comprise any of the features taught or disclosed herein, in accordance with various aspects of the present disclosure.

The one or more protrusions 84 may provide further stability to bone plate system 80. In some aspects, the one or more protrusions 88 may reinforce the plate 82. In some aspects, the one or more protrusions 84 may maintain alignment between the first and second or first and second bone portions. In some aspects, the plate 82 and the one or more protrusions 84 may be integrally formed. In some aspects, the plate 82 and the one or more protrusions 84 may be separately formed.

The one or more protrusions 84 can be oriented generally transverse relative to a keel structure 84 of the system 80. In some embodiments, the protrusion(s) 84 can be oriented generally perpendicular relative to the keel structure. Additionally, the protrusion(s) 84 can be formed to be continuous or separate from the keel structure. The protrusion(s) 84 can also define a height that is different from the height of the keel structure. For example, the protrusion(s) 84 can extend at a greater or lesser distance from the bone engaging surface 86 than the keel structure.

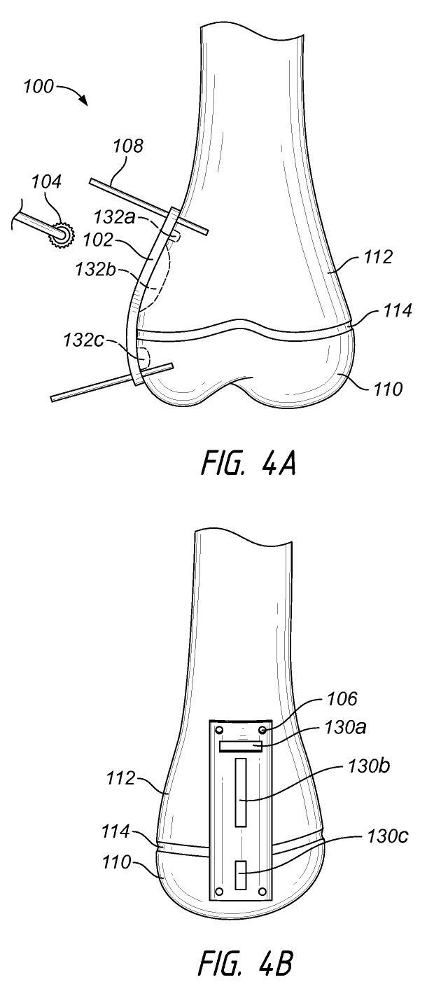

FIGS. 4A and 4B illustrate an example of a jig system 100 used for fastening the a plate to bone portions 110, 112, which can be first and second bone portions, in accordance with various aspects of the present disclosure. FIG. 4A shows a side view of the jig system 100, and FIG. 3B shows a top view of jig system 100. The jig system may comprise a jig 102, saw 104, and one or more fasteners 106. In some aspects, jig 102 may be used as a guide for saw 104 to cut a groove into bone portions 110, 112 such that the plate system can be fastened to bone portions 110, 112 with the keel structure and/or one or more protrusions fitting substantially within the groove(s). In some aspects, bone portions 110, 112 may define a growth plate, epiphyseal plate, or physis 114 therebetween.

Jig 102 may comprise one or more holes 106. In some aspects, jig 102 may be fastened to bone portions 110, 112 using fasteners 108. Holes 106 may receive fasteners 108 therethrough for fastening and securing jig 102 to bone portions 110, 112. For example, bone may be drilled through holes 106 to form channels in bone portions 110, 112 such that fasteners 108 may be inserted through holes 106 into the channels for fastening jig 102 to bone portions 110, 112. Fasteners 108 may comprise k wires, screws, nails, or other suitable fasteners known to those of skill in the art.

Jig 102 may also comprise one or more slots 130 (e.g., as shown by slot 130a, 130b, and 130c). Slots 130 may permit cutting of bone through a respective slot. For example, saw 104 may be used to cut bone through slots 130 to form grooves 132 (e.g., as shown by groove 132a, 132b, and 132c). In some aspects, slots 130 may span physis 114 (e.g., slot 130a) when jig 102 is fastened to bone portions 110, 112.

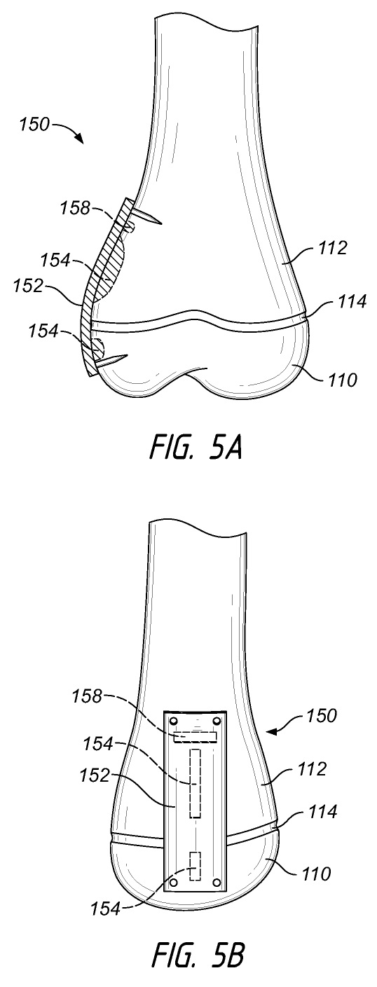

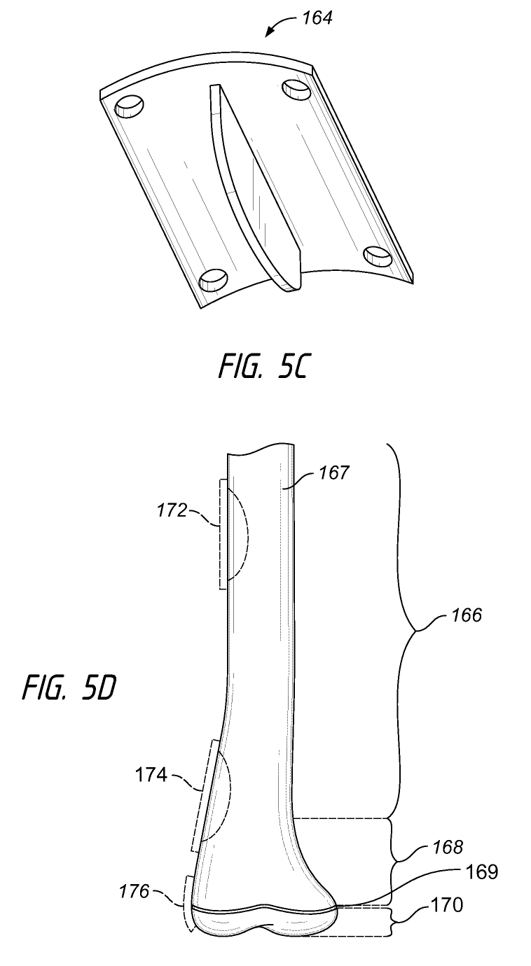

Following the preparation of bone portions 110, 112 using the jig system 100, a bone plate system can then be attached to the bone portions 110, 112. FIGS. 5A and 5B illustrate an example of a bone plate system 150 with a plate 152 fastened to bone portions 110, 112, in accordance with various aspects of the present disclosure. FIG. 5A shows a side view of bone plate system 150, and FIG. 5B shows a top view of bone plate system 150. The plate 152 may be fastened to bone portions 110, 112 such that a keel structure 154 fits substantially within groove 132a of FIG. 4A. In some aspects, the plate 152 may be fastened to bone portions 110, 112 such that a protrusion(s) 158 may fit substantially within grooves 132b and 132c. The keel structure 154 may reduce rotational, torsional and/or translational forces acting on bone portions 110, 112 during the growth of the bone.

As noted above, the methods and apparatuses disclosed herein can aid in realigning limbs of children, e.g., by affixing a plate to the diaphysis, metaphysis, and/or across the physis to the epiphysis. FIGS. 5A-B illustrate that in some embodiments, the plate can be affixed to the epiphysis (bone portion 110) and to the metaphysis (bone portion 112) and span the physis 114. Such an embodiment reflects the ability of a bone to grow at its ends, generally around the growth plate or physis 114.

With reference to FIGS. 5C-D, in accordance with some embodiments, methods and systems are also provided that enable a plate 164 to be affixed only in a diaphysis region 166 of a bone 167. However, the plate 164 can also be affixed only in a metaphysis region 168 of the bone 167 (see e.g., plate position 172 in FIG. 5D). Further, in some embodiments, the plate 164 can be affixed to the bone 167 and extend across both the diaphysis region 166 and metaphysis region 168 of the bone 167 (see e.g., plate position 174 in FIG. 5D). Furthermore, the plate 164 can be affixed to the bone 167 and extend across the metaphysis region 168, a physis region 169, and an ephiphysis region 170 of the bone 167 (see e.g., plate position 176 in FIG. 5D).

As noted above, any of the long bones of the body can be targeted using the systems and methods disclosed herein, such as the femur, humerus, radius, ulna, tibia, and fibula. These methods and apparatuses can be used to align portions of long bones, correcting issues including, for example, cubitus varus, knee valgus, knee varus, oblique plane, flexion deformity, recurvatum, and ankle valgus. Further, as also disclosed herein, any of the plate embodiments, e.g., the plates 152, 164 (using a single, split, or multiple keel structure) can be used in any of the plate positions 172, 174, 176 illustrated in FIG. 5D.

Further, in accordance with some embodiments, the positions 172, 174, 176 can be determined based on the curvature or shape of the bone 167. For example, a plate can be positioned on a first side of a bone in order to generally restrain growth of the bone on the first side while allowing the bone to grow uninhibited on the second side. In such an embodiment, the first side can have a generally convex bend or shape while the second side has a generally concave bend or shape. Accordingly, inhibiting growth on the first side while permitting growth on the second side can allow the bone to grow towards a generally straight configuration.

In accordance with some embodiments, the keel structure of the bone plate system can also comprise a stabilizing mechanism, such as a fin structure. The stabilizing mechanism can extend at least partially along the keel structure. In some embodiments, the stabilizing mechanism can comprise a pair of fins that extend from opposing sides of one or more separate or interconnected keels of the keel structure.

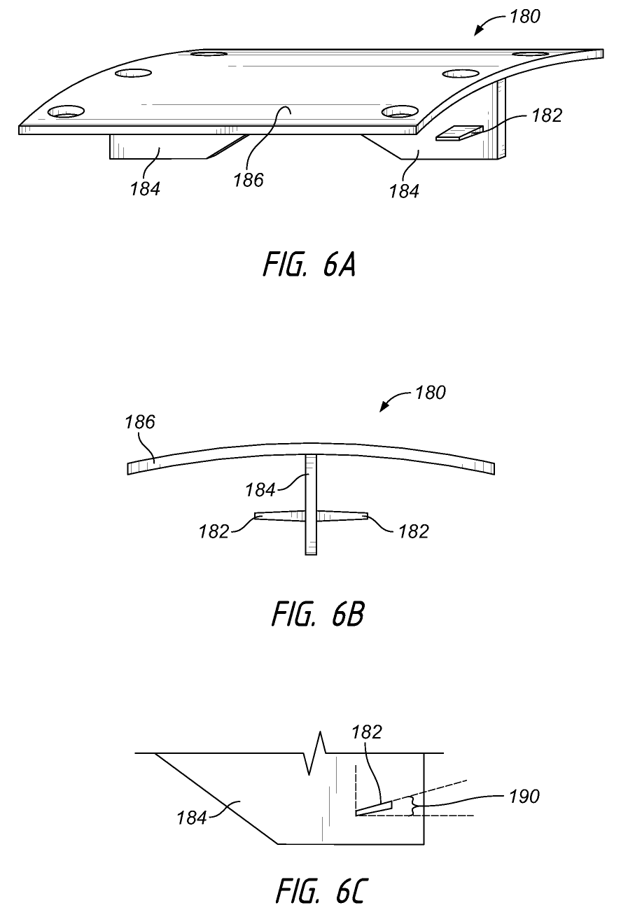

For example, referring now to FIGS. 6A, 6B, and 6C, an example is provided of bone plate system 180 with fin 182 coupled to a keel structure 184 of a plate 186, in accordance with various aspects of the present disclosure. FIG. 6A shows a general view of bone plate system 180. FIG. 6B shows a front view of bone plate system 180, and FIG. 6C shows a side view of the keel structure 184 and fin 182. Fin 182 may provide further stability the plate 186 when the plate 186 is fastened to one or more bone portions. Furthermore, having fin 182 may result in bone portions becoming aligned with each other and compressing against each other when the plate 186 is being fastened to the bone portions. Fin 182 may project from the keel structure 184 and have a fin angle 190 with respect to the plate 186 (such as a normal passing through the plate 186, as illustrated in FIG. 6C). For example, a long axis of fin 182 and a long axis of the plate 186 may be substantially transverse or non-parallel relative to each other.

In some aspects, fin 182 and the keel structure 184 may be integrally formed. In some aspects, fin 182 may comprise at least one of steel or titanium. In some aspects, fin 182 may comprise porous metal. For example, fin 182 may comprise at least one of trabecular metal or biofoam. Fin 182 may be substantially orthogonally coupled to the keel structure 184. The fin 182 can also be flexible and/or deflectable relative to the keel structure 184. For example, the fin 182 can deflect upwards towards the keel structure 184 and the plate 186 during insertion of the keel structure 184 into a groove formed in a bone portion. However, in some embodiments, downward deflection of the fin 182 away from the plate 186 can be generally prevented beyond a certain orientation, such as when the fin 182 is oriented generally perpendicular relative to the keel structure 184, absent a superseding force, such as that applied when the plate is removed.

In use, the fin 182 may act to provide alignment between bone portions 110, 112 and/or compression of bone portions 110, 112 against each other in order to achieve a desired orientation of the bone portions 110, 112 during growth. In some aspects, adjusting the fin angle 190 may correspondingly allow fin 182 to move farther in a direction either away or towards the plate 184 as growth between the bone portions 110, 112 occurs and the plate 186 moves relative to the bone portions 110, 112. Accordingly, the general orientation and/or compression of the plate 186 relative to the bone portions 110, 112 and/or the general orientation and/or compression of the bone portions 110, 112 relative to each other can be modified over time to compensate for anticipated bone portion migration, bone portion length, or changes in the angle of bone correction.

According to certain aspects of the present disclosure, the plate and the keel structure may be separate from one another. The plate may comprise one or more keel slot(s), and once the plate is fastened to bone portions, the keel slot(s) may permit cutting of bone through the keel slot(s) to generate a groove(s). In such embodiments, the keel structure, which can be separate from the plate, may then be inserted into the groove and attached to the plate in the keel slot(s).

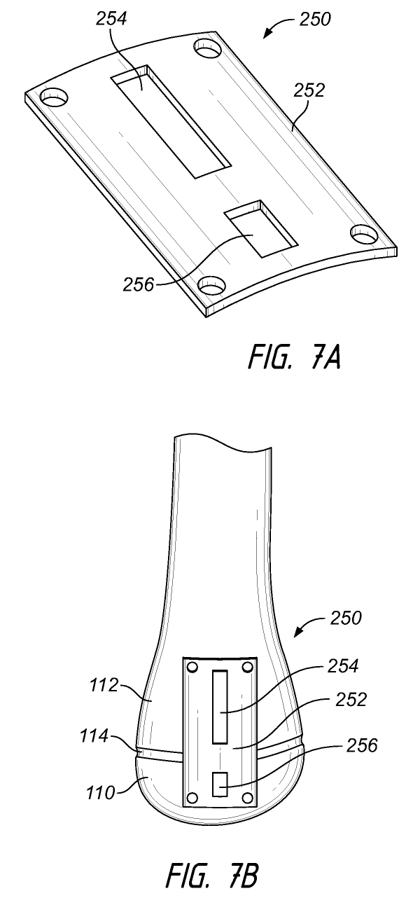

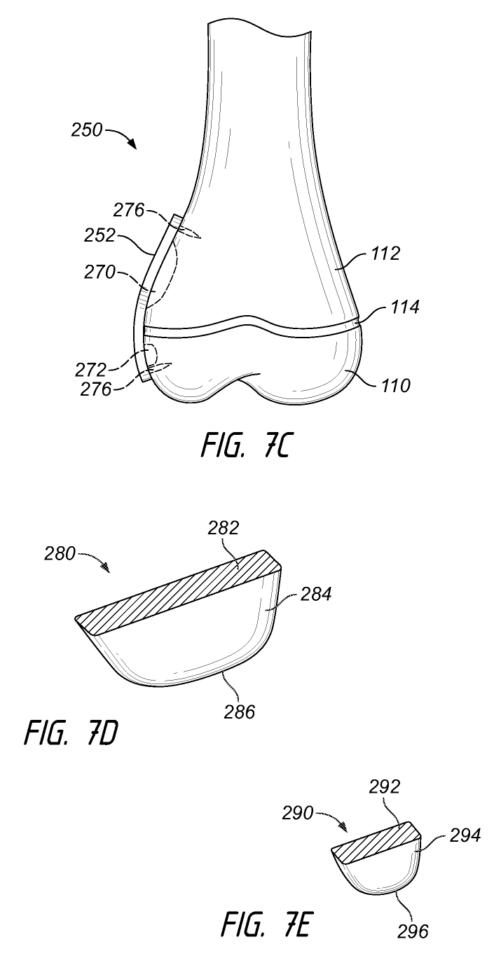

For example, FIG. 7A illustrates a perspective view of an example of a bone plate system 250 having such a configuration, in accordance with various aspects of the present disclosure. As illustrated in FIG. 7A, the bone plate system 250 comprises a plate 252, which is configured to be fastened to at least one bone portion (e.g., bone portions 110, 112). The plate 252 comprises a pair of keel slots 254, 256. FIG. 7B illustrates a top view of the bone plate system 250 attached to the bone portions 110, 112. As illustrated, the keel slots 254, 256 can be positioned over the respective bone portions 110, 112. According to certain aspects, the plate to 52 can span the physis 114 when fastened to bone portions 110, 112 while ensuring that the keel slots 254, 256 do not overlap the physis 114. When the grooves have been cut through the keel slots 254, 256, first and second keels can be inserted through keel slots 254, 256 into bone portions 110, 112.

FIG. 7C illustrates a side view of bone plate system 250, in accordance with various aspects of the present disclosure. As shown, the bone plate system 250 also comprises first and second keels 270, 272, which are configured to extend into bone portions 110, 112 through keel slots 254, 256 when the plate 252 is fastened to bone portions 110, 112 (e.g., using one or more fasteners 276). The plate system 250 can be affixed to a bone using fasteners 276, such as k wires, screws, nails, or other suitable fasteners known to those of skill in the art. As with the fasteners discussed above, in some embodiments, the fasteners 276 can be screws that affix the plate 252 to the bone and are configured to separate or diverge like a hinge, allowing the physis 114 to expand more rapidly on one side of the bone as the bone grows. This hinge action allows the plate 252 to generally permit growth on the unrestrained side of the bone (opposite the side on which the plate is applied) while compressing or restraining growth on the side of the bone to which the plate 252 is applied. For example, a curvature in a bone can be reduced and/or eliminated to correct bone deformity. In order to do so, the plate can be placed on the side of the bone that bows outward or appears convex, such that the opposite side (which bows inwardly or appears concave) can then grow in the direction of the keel longitudinal axis to promote growth of the bone toward a linear or straight axial configuration. Thereafter, normal bone growth can resume on both sides of the bone, e.g., on both sides of the physis.

The keel structure, e.g., the first and second keels 270, 272, may assume a variety of shapes. FIG. 7D illustrates an example of the keel structure 280, in accordance with various aspects of the present disclosure. As shown in FIG. 7D, the keel structure 280 comprises head region 282 and body region 284. A tip 286 of the keel structure 280 may be curved. However, tip 286 may be sharp or assume other suitable shapes. As illustrated in FIGS. 7A-C, a first keel slot 254 can be longer than a second keel slot 256. Accordingly, as shown in FIGS. 7D-7E, the keel structure 280 used for the first keel slot 254 can be longer than a keel structure 290 used for the second keel slot 256. Accordingly, the dimensions of the head regions 282, 292, the body regions 284, 294, and the tips 286, 296 can be varied according to the length of the corresponding keel slot and/or the bone shape. For example, a clinician can be provided with a variety of keel structures that can be used in response to patient bone parameters, thus providing the clinician with flexibility to specifically tailor the bone plate system to the needs of the patient.

According to various aspects of the present disclosure, the plate can comprise one or more locking assemblies. For example, if the plate has two keels, as illustrated in FIG. 7A, the plate can also comprise two respective locking assemblies, one for each keel. In some aspects, the locking assembly is configured to substantially prevent a keel structure from dislodging from the plate and/or bone portion when the keel structure extends into bone portion through a keel slot. In some aspects, the locking assembly is configured to substantially lock the keel structure to the plate when the keel structure extends into bone portion through keel slot. In some aspects, the term “lock” may include its ordinary meaning and/or may include secure, fasten, or some other manner in which the keel structure couples to the plate without the use of a key or a code.

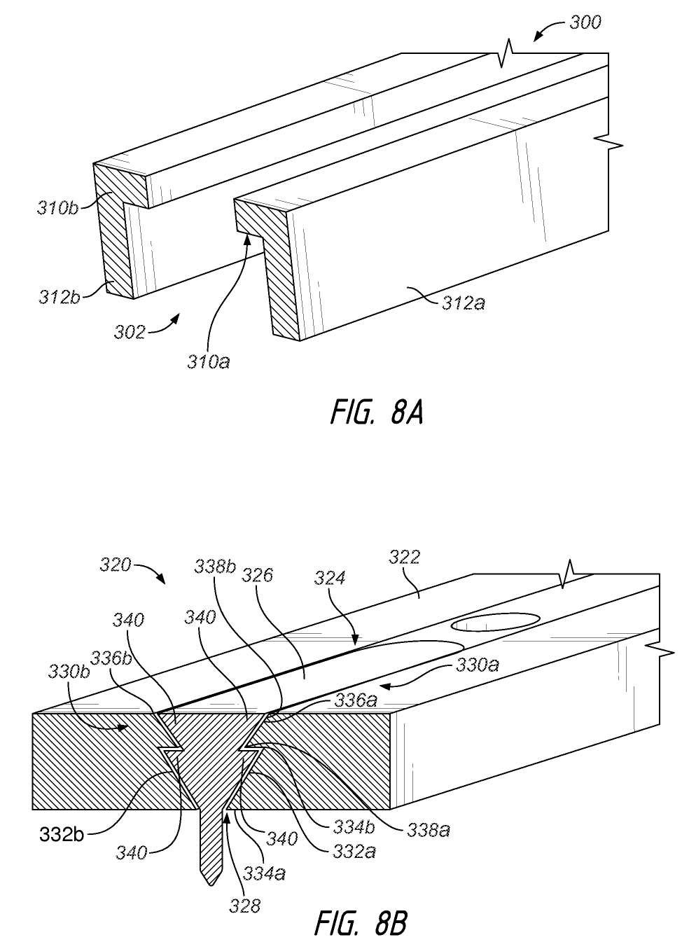

FIG. 8A illustrates a partial cross-section of a locking assembly 300, in accordance with an aspect of the present disclosure. The locking assembly 300 can comprise a keel slot 302 and lip regions (e.g., 310a and 310b) extending along respective walls (e.g., 312a and 312b) of the keel slot 302. In some aspects, wall 312a is opposite wall 312b. In some aspects, the locking assembly is configured to flex between a first state and a second state. Lip regions 310a and 310b are farther apart from one another in the first state than when lip regions 310a and 310b are in the second state. Thus, lip regions 310a and 310b may be flexed apart from one another to allow a keel structure to be inserted into keel slot 302. Although only one lip region is shown per wall of keel slot 302, the locking assembly may have any suitable number of lip regions extending along a wall of keel slot 302.

According to certain aspects, lip regions 310a and 310b may be angled from respective walls 312a and 312b at various suitable angles. For example, lip regions 310a and 310b may be angled toward one of the bone portions 110, 112 at less than or equal to 90 degrees from respective walls 312a and 312b when the plate is fastened to bone portions 110, 112. Such a configuration may facilitate one-way insertion of a keel structure into the keel slot 302, lock the keel structure to the plate when the keel structure extends into one of the bone portions through the keel slot 302, and/or prevent the keel structure from dislodging from the plate and/or bone portion when the keel structure extends into the bone portion through the keel slot 302.

FIG. 8B illustrates a cross-sectional view of another example of a locking assembly, in accordance with various aspects of the present disclosure. FIG. 8B illustrates a plate assembly 320 that comprises a plate 322, a locking assembly 324 integrated into the plate 322, and a keel structure 326 that can be inserted into the locking assembly 324.

The locking assembly 324 can comprise a wall 332a that may be slanted such that keel slots 254, 256 is narrower at edge 334a than at edge 334b, wherein edge 334a is closer to bone portions 110, 112 than edge 114b when the plate 322 is fastened to bone portions 110, 112. In some aspects, keel-engaging side 336a of lip region 330a is slanted such that keel slot 328 is narrower at edge 338a than at edge 338b, wherein edge 338a is closer to bone portions 110, 112 than edge 338b when the plate 322 is fastened to bone portions 110, 112. In some aspects, wall 332a and keel-engaging side 336a are slanted at substantially the same angle. In some aspects, the keel structure 326 comprises one or more ridges 340 extending along a first side of the keel structure 326. The one or more ridges 340 may be sized to fit against wall 332a and lip region 330a. Such a configuration may facilitate one-way insertion of the keel structure 326 into keel slot 328, lock the keel structure 326 to the plate 322 when the keel structure 326 extends into bone portions 110, 112 through keel slot 328, and/or prevent the keel structure 326 from dislodging from the plate 322 and/or bone portions 110, 112 when the keel structure 326 extends into bone portions 110, 112 through keel slot 328. A similar configuration may be implemented on wall 332b, lip region 330b, keel-engaging side 336b, and/or a second side of the keel structure 326 opposite the first side of the keel structure 326.

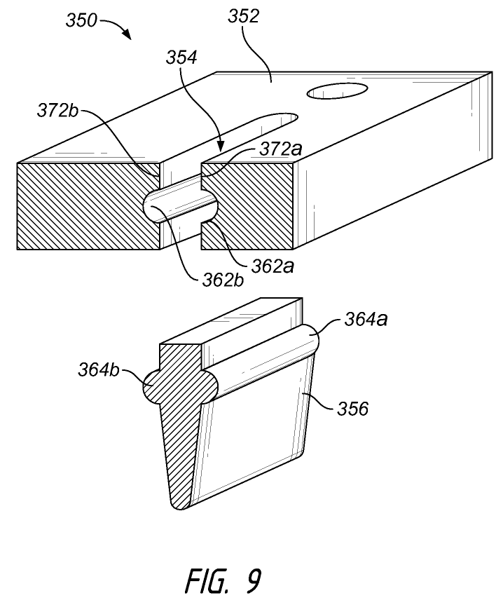

FIG. 9 illustrates a cross-sectional view of another example of a locking assembly, in accordance with various aspects of the present disclosure. FIG. 9 illustrates a plate assembly 350 that comprises a plate 352, a locking assembly 354 integrated into the plate 352, and a keel structure 356 that can be inserted into the locking assembly 354.

The locking assembly 354 can comprise a keel slot 358 and groove regions 362a and 362b extending along respective walls 372a and 372b of keel slot 358. In some aspects, the keel structure 326 comprises projection regions 364a and 364b. Projection region 364a extends along a first side of the keel structure 356, and projection region 364b extends along a second side of the keel structure 356 that is opposite the first side. Projection regions 364a and 364b are sized to fit within respective groove regions 362a and 362b when the keel structure 356 extends into bone portions 110, 112.

In some aspects, walls 372a and 372b may be flexed apart from one another to allow the keel structure 326 to be inserted into keel slot 358 such that projection regions 364a and 364b fit within respective groove regions 362a and 362b. Such a configuration may lock the keel structure 326 to the plate 322 when the keel structure 326 extends into a bone portion through the keel slot 358 and/or prevent the keel structure 326 from dislodging from the plate 322 and/or bone portion when the keel structure 326 extends into the bone portion through keel slot 358. Although only a single groove region is shown on a wall of keel slot 358, any suitable number of groove regions may be implemented on a wall of keel slot 358. Although only a single projection region is shown on a side of the keel structure 326, any suitable number of projection regions may be implemented on a side of the keel structure 326.

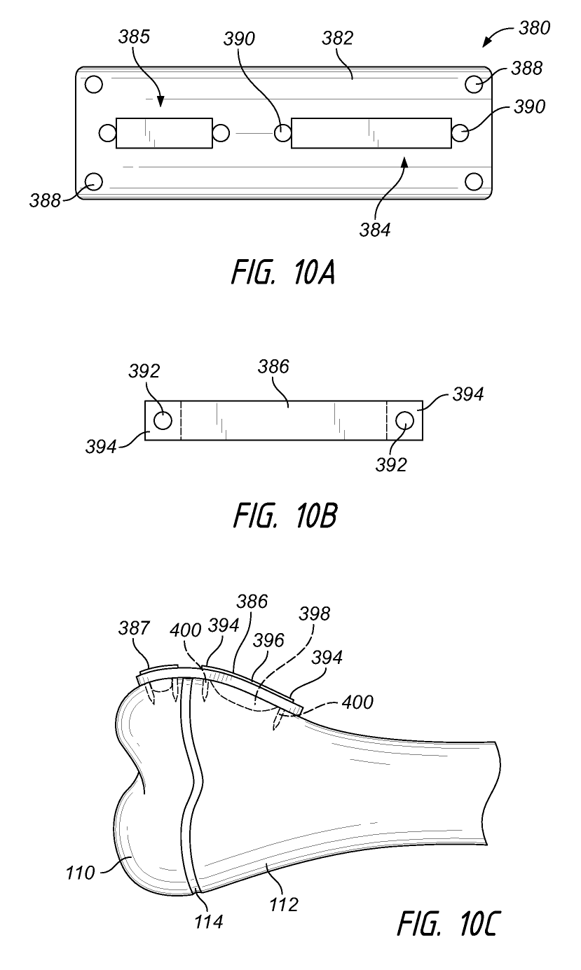

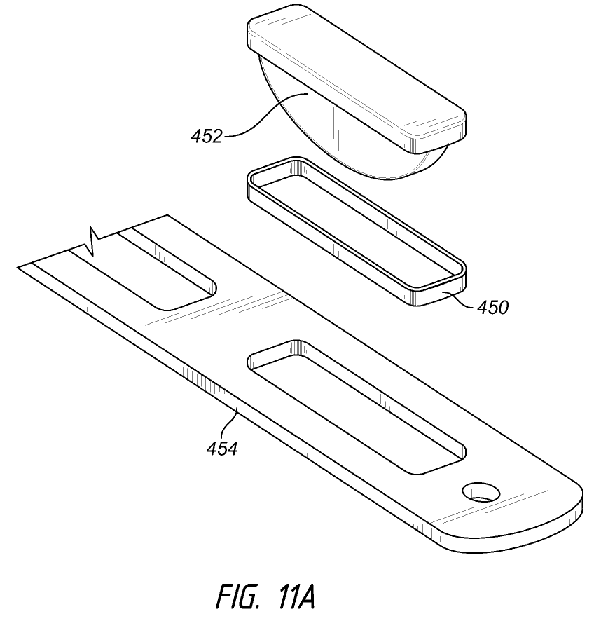

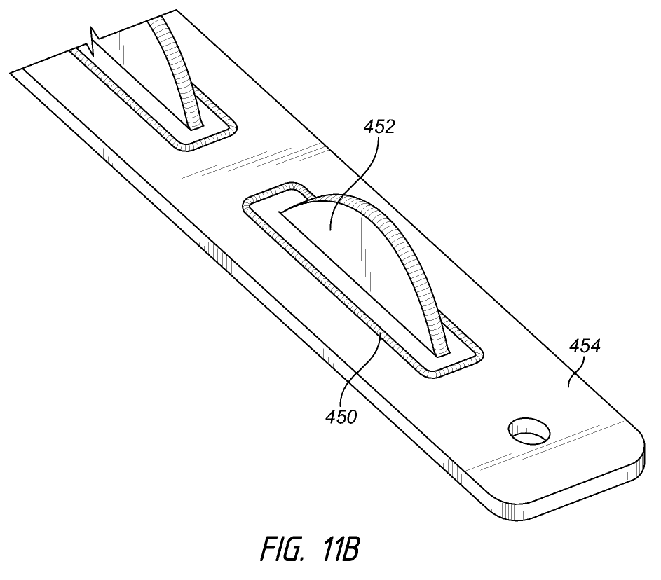

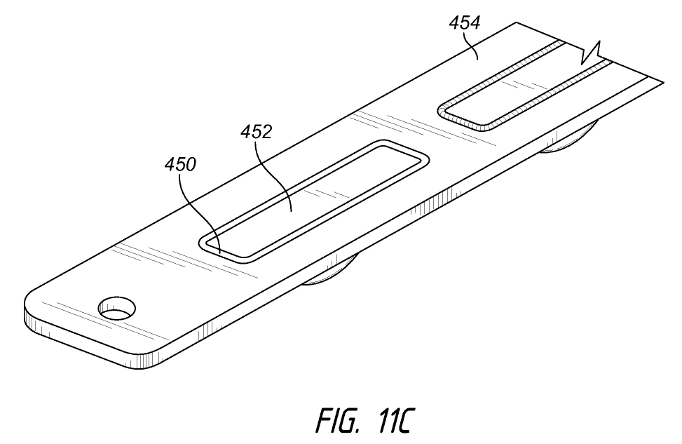

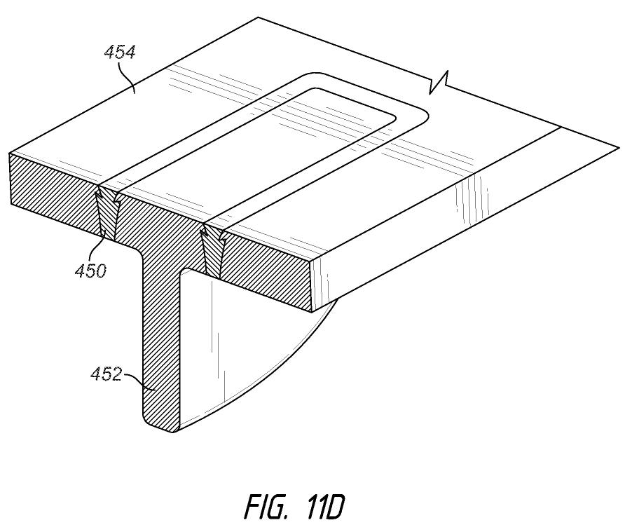

FIGS. 10A, 10B, and 10C illustrate another example of the locking assembly, in accordance with various aspects of the present disclosure. FIG. 10A-B illustrate a plate assembly 380 that comprises a plate 382, a locking assembly 384 integrated into the plate 382, and a keel structure 386 that can be inserted into the locking assembly 354.

In a top view of the plate 382, as shown in FIG. 10A, the locking assembly comprises one or more fastener holes 390 formed on the plate 382. In a top view of the keel structure 386, as shown in FIG. 10B, the keel structure 386 comprises one or more corresponding fastener holes 392. The one or more fastener holes 390 and 392 may each receive a fastener therethrough for fastening the keel structure 386 to the plate 382 when the keel structure 386 extends into bone portion 112.

According to certain aspects, the keel structure 386 comprises one or more tab regions 394. A corresponding fastener hole 392 is formed on each of the one or more tab regions 394. As shown in FIGS. 10A, 10B, and 10C, the one or more tab regions 394 extend from a head region 396 and are configured to engage the one or more fastener holes 390 of the plate 382 when the keel structure 386 extends into the bone portion 112. For example, the one or more tab regions 394 may be positioned such that the fastener holes 390 of the plate 382 are aligned with the one or more corresponding fastener holes 392 of the keel structure 386. In this way, one or more fasteners 400 may be inserted through the one or more fastener holes 390 and the one or more corresponding fastener holes 392 to lock the keel structure 386 to the plate 382 and/or fasten the plate 382 to the bone portion 112. In some aspects, other fasteners may be used to fasten the plate 382 to the bone portion 112 (e.g., using holes 392 on the plate 382).

Additionally, as illustrated in FIG. 10A, the plate 382 can also comprise a second locking assembly 385 configured to receive a second keel structure 387. The second keel structure can be configured and used in a manner similar to the keel structure 386. Accordingly the above discussion with respect to the locking assembly 384 shall apply equally to the locking assembly 385 and will not be repeated herein for brevity.

In some aspects, the keel structure may provide additional stability by contacting perimuscular fascia or other soft tissue when the plate is fastened to bone portions 110, 112. In some aspects, a body region of the keel structure can be shaped appropriately such that the keel structure does not penetrate or damage the perimuscular fascia or other soft tissue. For example, the body region may taper away from the plate. In some aspects, the body region (e.g., including its tip) can be curved and/or rounded off.

In some embodiments, the keel structure can be integral with or formed from a continuous piece of material as the plate. However, the keel structure may be a separate component from the plate in certain situations, such as when the plate is already fastened to bone portions and the keel structure is added to the plate to provide additional stability. In these situations, the locking assembly as described previously (e.g., with respect to FIGS. 7A-7E, 8A-8B, 9, and 10A-10C) may be used in a similar manner to lock the keel structure to the plate.

The structures described in this application (e.g., plates, the keel structures, locking assemblies, jigs, fins, spacers, fasteners, etc.) may be made of any suitable material, such as stainless steel and/or titanium. According to certain aspects, a liner may be used between the structures to substantially prevent contact from one another (e.g., metal-to-metal contact). In some aspects, the liner may be an intervening polymer portion. In some aspects, the liner may be made of a biocompatible, deformable, and/or fatigue resistant polymer that increases the contact area between the structures (e.g., between the keel structure and the plate). For example, the intervening polymer portion may comprise polyether ether ketone (PEEK) or other suitable materials. The shape of the liner may conform to any of the areas of contact between the structures. In some aspects, the liner may comprise a removal member that is configured to facilitate its removal from the areas of contact. For example, the liner may comprise a tooth, a divot, a lip, or some other suitable mechanical member to facilitate removal.

According to certain aspects, the liner may increase the contact area between the keel structure and the plate. For example, the liner may be placed in a keel slot between the keel structure and the plate to reduce mechanical micro-motion and improve fatigue resistance. FIGS. 11A-11D illustrate various views of a liner 450 being used to prevent contact between a keel structure 452 and a plate 454, in accordance with various aspects of the present disclosure. FIG. 11A is an exploded view of the plate 454, liner 450, and the keel structure 452. FIG. 11B is a bottom perspective view of the plate 454, liner 450, and the keel structure 452. FIG. 11C is a top perspective view of the plate 454, liner 450, and the keel structure 452. FIGS. 11D is a cross-sectional view of the plate 454, liner 450, and the keel structure 452. As shown in this figure, liner 450 may conform to the surfaces of the keel structure 452 and the plate 454, even if the plate 454 comprises various lip regions and the keel structure 452 comprises various ridges (e.g., as described with respect to FIGS. 8A and 8B). In this embodiment, the liner 450 can comprise an angled or slanted tooth-type engagement structure and the keel structure 452 can comprise a corresponding angled or slanted tooth-type engagement structure. The engagement structures of the liner 450 and the keel structure 452 can comprise protrusions, recesses, or apertures.

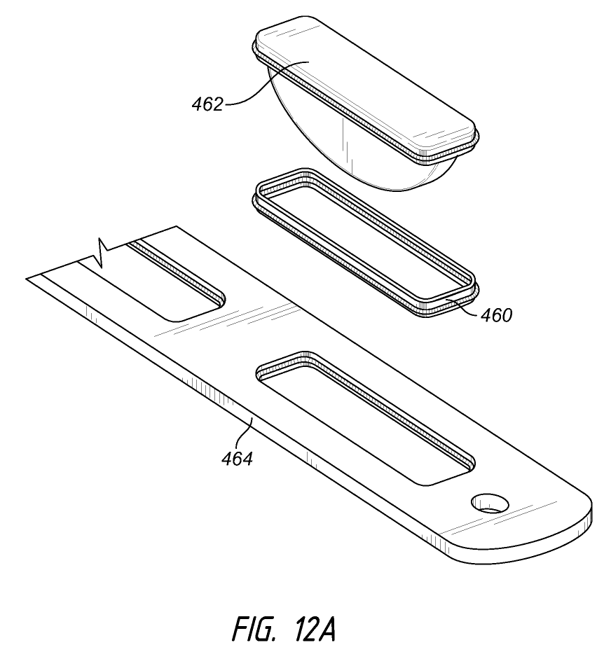

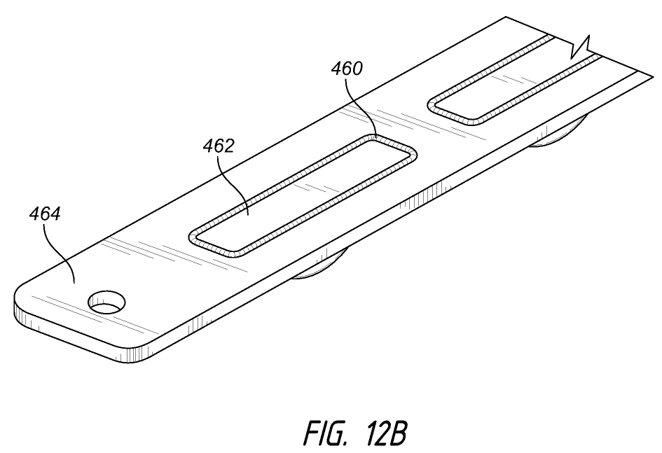

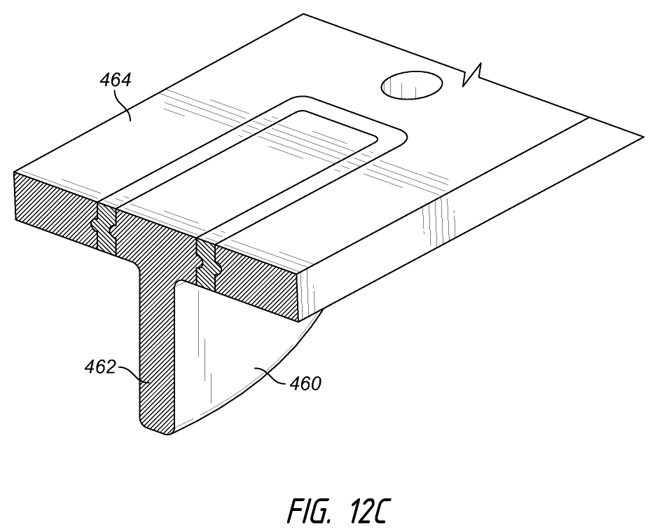

FIGS. 12A, 12B, 12C, and 12D illustrate various views of liner 460 being used to prevent contact between the keel structure 462 and the plate 464, in accordance with various aspects of the present disclosure. FIG. 12A is an exploded view of the plate 464, liner 460, and the keel structure 462. FIG. 12B is a top perspective view of the plate 464, liner 460, and the keel structure 462. FIGS. 12C is a cross-sectional view of the plate 464, liner 460, and the keel structure 462. As shown in this figure, liner 460 may conform to the surfaces of the keel structure 462 and the plate 464, even if the plate 464 comprises various groove regions and the keel structure 462 comprises projection regions (e.g., as described with respect to FIG. 23). In this embodiment, the liner 460 can comprise a rounded or elongate tubular engagement structure and the keel structure 462 can comprise a corresponding rounded or tubular elongate engagement structure. These engagement structures of the liner 460 and the keel structure 462 can comprise protrusions, recesses, or apertures.

As illustrated in FIGS. 11A-11D and 12A-C, the liner can be used for multiple keel structures on the plate. Further, although FIGS. 11A-11D and 12A-C illustrate slanted teeth and rounded bumps, respectively, the engagement structure used between the liner and the plate and keel structure can be modified to incorporate a variety of structures, whether used in a pair or in combination with other structures.

The methods and systems described herein may be applied anywhere in a skeleton and in particular, on long bones at the physis. For example, the methods and systems described herein may be applied to the femur. In another example, the plate systems as described herein may be used on the humerus, on the tibia, on the fibia, or on any other long bone of the skeleton. The methods and systems described herein are not limited to human skeletons but may also be applied to animal skeletons as well.

The foregoing description is provided to enable a person skilled in the art to practice the various configurations described herein. While the present invention has been particularly described with reference to the various figures and configurations, it should be understood that these are for illustration purposes only and should not be taken as limiting the scope of the invention.

There may be many other ways to implement the invention. Various functions and elements described herein may be partitioned differently from those shown without departing from the scope of the invention. Various modifications to these configurations will be readily apparent to those skilled in the art, and generic principles defined herein may be applied to other configurations. Thus, many changes and modifications may be made to the invention, by one having ordinary skill in the art, without departing from the scope of the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

In some aspects, the phrase “substantially” as used herein refers to being within at least 99%. For example, when the keel structure of a plate fits substantially within a groove, the keel structure may fit within at least 99% of the groove. In some aspects, the phrase “substantially” as used herein refers to being within at least 95%. In some aspects, the phrase “substantially” as used herein refers to being within at least 90%. In some aspects, the phrase “substantially” as used herein refers to being within at least 80%. In some aspects, the phrase “substantially” as used herein refers to being within at least 70%. In some aspects, the phrase “substantially” as used herein refers to being within at least 60%. In some aspects, the phrase “substantially” as used herein refers to being within at least 50%. In some aspects, the phrase “substantially” as used herein is given its ordinary meaning

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” The term “some” refers to one or more. All structural and functional equivalents to the elements of the various configurations described throughout this disclosure that are known or later come to be known to those of ordinary skill in the art are expressly incorporated herein by reference and intended to be encompassed by the invention. Moreover, nothing disclosed herein is intended to be dedicated to the public regardless of whether such disclosure is explicitly recited in the above description.

The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

What is claimed is:

1. A method for controlling growth of a growing bone, the bone having a first side and a second side opposite the first side, the method comprising applying a bone fixation plate to the first side of the bone, the plate comprising at least one keel having a longitudinal axis, the plate being applied such that when affixed to the bone, linear bone growth occurs on the second side of the bone in the direction of the keel longitudinal axis while bone growth is inhibited on the first side.

2. The method of claim 1, wherein when the plate is applied to the bone, the bone comprises a curvature and the plate is configured to permit growth along the second side of the bone to change the bone curvature.

3. The method of claim 1, wherein applying the plate comprises applying the plate to a metaphysis of the bone.

4. The method of claim 1, wherein applying the plate comprises applying the plate to a diaphysis of the bone.

5. The method of claim 1, wherein applying the plate comprises applying the plate to both a metaphysis and a diaphysis of the bone.

6. The method of claim 1, wherein applying the plate comprises applying the plate to both a metaphysis and an epiphysis of the bone.

7. The method of claim 1, wherein the keel comprises first and second keel portions, and the bone comprises a physis, and wherein the method further comprises applying the plate such that the first and second keel portions are positioned on opposing sides of the physis.

8. A method for stabilizing first and second bone portions of a person whose bones are growing, the first and second bone portions being separated by a physis, the method comprising:
positioning a bone fixation plate to extend from the first bone portion to the second bone portion across the physis;
affixing a first portion of the plate to the first bone portion, the plate comprising a first keel portion extending into the first bone portion; and
affixing a second portion of the plate to the second bone portion, the plate comprising a second keel portion extending into the second bone portion, the first and second keels defining a keel longitudinal axis,
wherein when affixed to the bone, the plate is configured to permit growth in the direction of the keel longitudinal axis.

9. The method of claim 8, wherein affixing the plate first portion to the first bone portion comprises inserting a screw through a first hole formed in the first portion, and wherein the affixing the plate second portion to the second bone portion comprises inserting a screw through a second hole formed in the second bone portion.

10. The method of claim 8, wherein positioning the bone fixation plate comprises orienting the first and second keel portions to extend generally along the longitudinal axis of the bone such that the plate limits growth in the direction of the bone longitudinal axis.

11. The method of claim 8, wherein affixing the plate first portion to the first bone portion comprises extending the first keel portion into only the cortex of the first bone portion, and wherein affixing the plate second portion to the second bone portion comprises extending the second keel portion into only the cortex of the second bone portion.

12. A bone fixation system, for stabilizing first and second portions of a bone separated by a physis, comprising:
a bone fixation plate having first and second portions configured to extend across the physis and be fastened to the first and second bone portions; and
a split keel structure having a first keel portion extending from the plate first portion and a second keel portion extending from the plate second portion, the second keel portion separate from the first keel portion, the first and second keel portions being configured to extend into the first and second bone portions.

13. The system of claim 12, wherein the first and second keel portions are interconnected by an intermediate ridge.

14. The system of claim 12, wherein the first and second keel portions converge toward each other and taper toward the plate.

15. The system of claim 12, wherein the first and second keel portions are oriented parallel relative to each other.

16. The system of claim 12, wherein the first keel portion has a shorter longitudinal length than the second keel portion.

17. The system of claim 12, wherein the system comprises a first protrusion extending transversely relative to and adjacent to the first keel portion.

18. The system of claim 17, wherein the system comprises a second protrusion extending transversely relative to and adjacent to the second keel portion.

19. The system of claim 12, wherein the plate comprises a first keel slot, and the system further comprises a locking assembly, the first keel slot configured to engage with the first keel portion to prevent the first keel portion from dislodging from the plate when the first keel portion extends into the first bone portion through the first keel slot.

20. The system of claim 19, wherein the plate further comprises a second keel slot, the second keel slot configured to engage the second keel portion to prevent the second keel portion from dislodging from the plate when the second keel portion extends into the second bone portion through the second keel slot.
